(19) United States
(12) Patent Application Publication
(10) Pub. No.: US 2024/0091551 A1
(43) Pub. Date: Mar. 21, 2024

(54) DEVICES AND METHODS FOR LIGHT THERAPY

(71) Applicant: Curavi Medical Technologies, LLC, Miami, FL (US)

(72) Inventors: Jose Carlos CABANA, Miami, FL (US); Joaquin Daniel Brillembourg TORREALBA, Aventura, FL (US); Hector Mario DE MARTIN VIANA, Miami, FL (US); Eunice BATRES, South Miami, FL (US)

(21) Appl. No.: 18/468,521

(22) Filed: Sep. 15, 2023

Related U.S. Application Data
(60) Provisional application No. 63/376,066, filed on Sep. 16, 2022.

(51) Int. Cl. A61N 5/06; A61N 5/067; H01S 5/023; H01S 5/042

(52) U.S. Cl. CPC A61N 5/0613; A61N 5/067; H01S 5/023; H01S 5/042; A61N 2005/0626; A61N 2005/0645; A61N 2005/0652; A61N 2005/0663; A61N 2005/0664; H01S 2302/00

(57) ABSTRACT

Described herein are light therapy devices for treating pain. The devices generally include a plurality of light sources configured to stimulate photobiomodulation in tissue, and may be equipped with a safety mechanism configured to stabilize power and current distribution. Methods for treating pain using the light therapy devices are also described herein.

### CROSS-REFERENCE TO RELATED APPLICATIONS

This application claims priority to U.S. Provisional Application No. 63/376,066, filed on Sep. 16, 2022, which is hereby incorporated by reference in its entirety.

### FIELD

This application generally relates to light therapy devices for treating pain. The devices generally include a plurality of light sources configured to stimulate photobiomodulation in tissue, and may be equipped with a safety mechanism configured to stabilize power and current distribution. Methods for treating pain using the light therapy devices are also described herein.

### BACKGROUND

Pain is an uncomfortable feeling that oftentimes indicates something may be wrong in the body and may be classified as either acute or chronic. Acute pain is usually severe and short-lived, and is often a signal that your body has been injured. Chronic pain can range from mild to severe, is present for longer periods of time, and may often be the result of a disease requiring ongoing treatment.

Just as pain has various etiologies, there are several ways in which pain may be treated or managed. For example, medications administered orally or via injection (e.g., into a vein, joint, subcutaneously, or spinal area) may be used. With chronic pain, therapies such as acupuncture, TENS (Transcutaneous Electrical Nerve Stimulation), bioelectric therapy, and physical therapy may also be employed.

Another type of treatment that may be used for pain is Low Level Laser Therapy (LLLT). LLLT functions through the process of photobiomodulation. Photobiomodulation Therapy (PBMT) is a type of light therapy that utilizes non-ionizing forms of light sources, including lasers, LEDs, and broad-band light, in the visible and infrared spectrum. It is a process that may be beneficial in managing various conditions and body processes, including but not limited to the alleviation of pain or inflammation, and tissue regeneration. In some instances, PBMT has been used for hair loss prevention and hair regrowth, acne treatment, promoting collagen production, reducing wrinkles, fat loss, and stem cell production. The efficacy of treatment of these various conditions and for pain may be based on the wavelength of light used, the treatment protocol employed, or both. However, to date, there are no established parameters relating to the effectiveness and quality of light treatment, e.g., the specific wavelength of light, source of light, and/or the pattern of delivery of the light.

Accordingly, it would be useful to have new light therapy devices and methods for the treatment or management of pain and other conditions.

### SUMMARY

Described herein are light therapy devices configured to be worn by a subject. The light therapy devices may include a plurality of light sources electrically coupled to a printed circuit board. The plurality of light sources may be configured as an array having multiple rows, where each row of the multiple rows may include a power distribution channel at a first end of the printed circuit board. Additionally, the light therapy devices may include a safety mechanism comprising a fuse associated with the power distribution channel, and a network of bridge jumpers on the printed circuit board configured to stabilize current flow to the plurality of light sources.

The plurality of light sources may be attached to, or arranged on or within (e.g., embedded in) a flexible material and configured as a wearable garment for placement on various body areas. Exemplary flexible materials include without limitation, polyurethane, polyester, styrene-butadiene rubber, or a combination thereof. The size, shape, and type of garment, and the materials used may vary depending on the body area upon which the garment is to cover. The flexibility of the material may allow the garment to conform to the surface of the body area. The wearable garment may be a belt, band, brace, sling, sleeve, or other type of wrap configured to place the plurality of light sources against the surface, e.g., the skin, of the body area in which the subject is experiencing pain. Some variations of the wearable garment may include a flexible or malleable metal band. In these variations, the wearable garment may be configured to be worn around an extremity (e.g., arm or leg), a joint (e.g., wrist, elbow, ankle), or around the neck. A single flexible layer, e.g., a layer made from silicone or other pliable material, may be disposed over the plurality of light sources on the side of the garment opposite the flexible material. In some variations, two flexible layers may be used. The two flexible layers may comprise silicone. One silicone layer may be disposed over the plurality of light sources and the second silicone layer may be disposed on the flexible material.

Any suitable light source may be included in the light therapy devices. For example, light emitting diodes (LEDs) or laser diodes may be used. In some instances, it may be beneficial to employ a plurality of laser diodes. Each laser diode of the plurality of laser diodes may be configured to emit light at a wavelength ranging from about 300 nm to about 1200 nm, including all values and sub-ranges therein. For example, the wavelength may be about 300 nm, about 350 nm, about 400 nm, about 450 nm, about 500 nm, about 550 nm, about 600 nm, about 610 nm, about 620 nm, about 630 nm, about 640 nm, about 650 nm, about 660 nm, about 670 nm, about 680 nm, about 700 nm, about 710 nm, about 720 nm, about 730 nm, about 740 nm, about 750 nm, about 760 nm, about 770 nm, about 780 nm, about 790 nm, about 800 nm, about 810 nm, about 820 nm, about 830 nm, about 840 nm, about 850 nm, about 900 nm, about 950 nm, about 1000 nm, about 1050 nm, about 1100 nm, about 1150 nm, or about 1200 nm. In one variation, it may be useful for the laser diode to emit light at a wavelength between about 600 nm to about 810 nm. In another variation, it may be useful for the laser diode to emit light at a wavelength of 650 nm. In a further variation, it may be useful for the laser diode to emit light at a wavelength of 808 nm.

Additionally, any suitable number of laser diodes may be included in the light therapy devices. The number of laser diodes included may depend on such factors as the size and shape of the garment, the particular condition being treated, and/or the body area being treated. In some variations, the number of laser diodes included may range from 4 to 220, including all values and sub-ranges therein. For example, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158,160, 162, 164, 166, 168, 170, 172, 174, 176, 178, 180, 182, 184, 186, 188, 190, 192, 194, 196, 198, 200, 202, 204, 206, 208, 210, 212, 214, 216, 218, or 220 laser diodes may be used. When the laser diodes are arranged as quartets, the number of quartets may generally range from 4 to 50. For example, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 quartets may be included in the light therapy devices. Although described as being arranged in quartets, it is understood that the laser diodes may be grouped in various other numbers.

Also described herein are methods for treating pain in a subject in need thereof including positioning a light therapy device on a body area of the subject, where the light therapy device may include a plurality of light sources electrically coupled to a printed circuit board, and delivering light from the plurality of light sources for a duration of time to the body area afflicted with pain. As described above, the printed circuit board may have a first end, and the plurality of light sources configured as an array having multiple rows, where each row of the multiple rows comprises a power distribution channel at the first end of the printed circuit board. The light therapy devices may further include a safety mechanism comprising a fuse associated with the power distribution channel, and a network of bridge jumpers on the printed circuit board configured to stabilize current flow to the plurality of light sources.

When treating pain, the duration of time in which light from each of the plurality of light sources may be delivered to a body area may range from about 5 minutes to about 30 minutes, including all values and sub-ranges therein. For example, the duration of time may be about 5 minutes, about 10 minutes, about 15 minutes, about 20 minutes, about 25 minutes, or about 30 minutes. In some instances, it may be beneficial for the duration of time to be about 12 minutes. A timer may be included in the light therapy devices. The timer may be pre-programmed with the duration of time for treating pain, and in some instances may also be manually adjusted after therapy has started. In other instances, the duration of time may be manually input by the subject. The pain being treated may be due to conditions such as, but not limited to, arthritis, inflammation, injury to the body area, or a combination thereof.

The light therapy devices may be placed on, and secured to or over, various body areas. For example, the body area may be a neck or a torso of the subject, or an upper back, a lower back, an abdomen, a shoulder, or a chest of the subject. The body area may also be a limb, e.g., an arm or a leg of the subject, or a joint area, e.g., an elbow, wrist, knee, or ankle of the subject.

Delivery or administration of light from the plurality of light sources for pain and other indications may occur once a day or multiple times a day. When delivered or administered multiple times a day, the time interval between treatment sessions may range between about 15 minutes to about 30 minutes, including all values and sub-ranges therein. For example, the time interval between session may be about 15 minutes, about 20 minutes, about 25 minutes, or about 30 minutes. The light therapy may also be repeated once a day for a plurality of days, or delivered multiple times a day for a plurality of days. The plurality of days may or may not be consecutive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts an exemplary light therapy device configured to be worn on the torso of a subject.

FIG. 2 depicts an exemplary silicone layer having pockets for holding a plurality of light sources.

FIG. 3 depicts an exemplary light therapy device including a first backing layer and a second material layer comprising a plurality of openings that allow transmission of light from the laser diodes.

FIGS. 4A-4C depict an exemplary light therapy device configured to be worn on the torso of a subject.

FIGS. 5A-5C depict an exemplary light therapy device configured to be worn on the neck of a subject.

FIGS. 6A and 6B depict an exemplary light therapy device configured to be worn on various joints of a subject. FIG. 6A shown the light therapy device being worn around the elbow of a subject, and FIGS. 6B and 6C show light therapy devices configured to be worn around the ankle of a subject.

FIG. 7 depicts an exemplary light therapy device configured to be worn around a single knee of a subject.

FIG. 8 depicts an exemplary light therapy device being worn around both knees of a subject.

FIG. 9 depicts an exemplary Automatic Power Control (APC) circuit design for a laser diode.

FIG. 10 depicts an exemplary circuit layout including a flexible 4×4 grid.

FIG. 11 depicts an exemplary safety mechanism including a fuse associated with a power distribution channel, and a network of bridge jumpers.

FIGS. 12A and 12B depict additional exemplary safety mechanisms including a plurality of fuses associated with power distribution channels and a network of bridge jumpers.

FIG. 13 depicts another exemplary light therapy device configured to be worn around a single knee of a subject.

FIG. 14 depicts an exemplary light therapy device configured to be worn on the hand of a subject.

FIGS. 15A and 15B are schematic representations of a circuit layout that may be used for an exemplary light therapy device configured to be worn on the back of a subject.

FIGS. 16A and 16B show an exemplary array of light sources configured to be worn on the back of a subject.

FIG. 17 shows an exemplary silicone layer having pockets for holding the light source array of FIGS. 16A and 16B.

FIG. 18A depicts a front view of an exemplary light therapy device configured to be wrapped around the back of a subject. FIG. 18B depicts a back view of the exemplary light therapy device of FIG. 18A.

FIG. 19A depicts a front view of the exemplary light therapy device of FIG. 18A in an open configuration. FIG. 19B depicts a back view of the light therapy device of FIG. 19A.

FIG. 20 shows an exemplary panel of the light therapy device of FIGS. 18A and 18B including the silicone layer of FIG. 17 therein.

FIG. 21 depicts the panel of the light therapy device of FIG. 20 including a plurality of light sources covered by a silicone layer.

FIG. 22 depicts an exemplary support member for the light therapy device of FIGS. 18A and 18B.

### DETAILED DESCRIPTION

Described herein are light therapy devices configured to be worn by a subject. The light therapy devices may be used to treat or manage various types of pain. For example, the light therapy devices described herein may be used for the treatment of back pain, neck pain, and/or joint pain (e.g., ankle, knee, elbow, or shoulder pain). The devices may include a plurality of light sources including, e.g., laser diodes, attached to, or disposed on or within a flexible material such that they are capable of conforming to the body area upon which the devices cover. The devices may be configured to operate at a low voltage such that risks associated with electricity and/or heat may be avoided. Additionally or alternatively, a safety mechanism may be included in the light therapy devices that may help prevent overheating of the device due to component failure at any given point. The devices described herein may be designed to be portable (e.g., battery operated) and easy to use. In some instances, the devices may be configured to be adjustable in size in order to improve fit of the device and/or the amount of coverage by the devices over an area of the subject's body.

### Light Therapy Devices

### Wearable Garments

The light therapy devices described herein may generally include a plurality of light sources attached to, or arranged on or within (e.g., embedded in) a material that is a flexible and/or an elastic material. The flexible and/or elastic material may be configured as a wearable garment for placement on various body areas in need of light treatment. Non-limiting examples of flexible materials may include polyurethane, polyester, silicone, styrene-butadiene rubber, or a combination thereof. Other materials that may be employed include without limitation, woven or non-woven fabrics, knitted materials, mesh, nylon, rayon, cotton, elastic materials such as neoprene, isoprene, and spandex, or combinations thereof. In one variation, the wearable garment may be made from polyurethane, polyester, and styrene-butadiene rubber. In some variations, the wearable garment may be made from a waterproof, water-resistant, or anti-microbial material. The waterproof, water-resistant, or antimicrobial properties of the materials may be provided as a coating on the material.

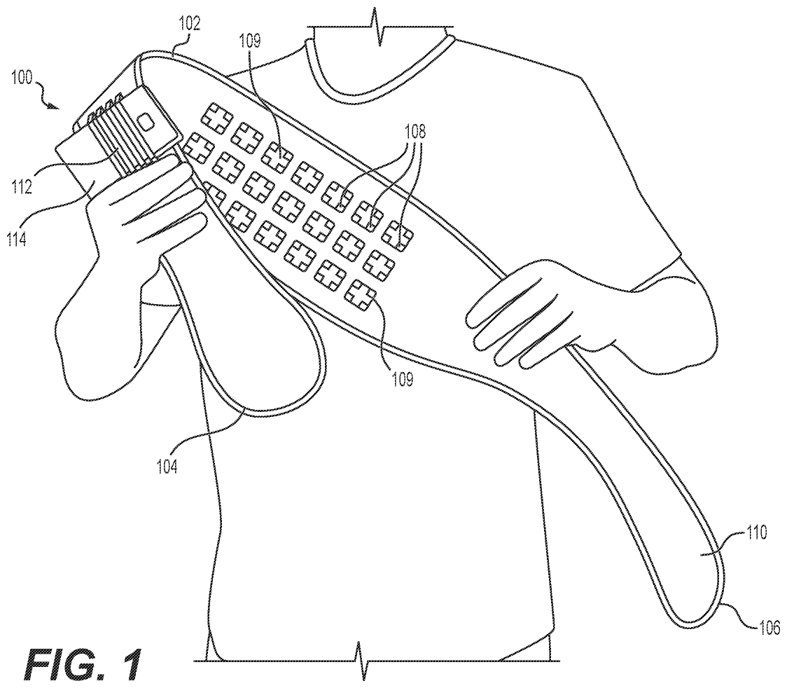

The wearable garment may include a single material layer or a plurality of material layers. When a plurality of material layers are employed, the light sources may be placed between layers of the material (e.g., between two, three, four, five layers of material). In some variations, the plurality of light sources may be placed between a first layer and a second layer of material. For example, the first layer of material may be a backing of the wearable garment, and a second layer of material (e.g., an inner layer that contacts the skin of a subject) may include a plurality of openings through which the light sources may be exposed. In these variations, a flexible layer, e.g., a layer made from silicone or a thermoplastic elastomer, may be disposed over the plurality of light sources but between the material layers such that the light sources are held in place within the garment. Exemplary thermoplastic elastomers include without limitation, styrenic block copolymers, polyolefin blends, elastomeric alloys, thermoplastic polyurethanes, thermoplastic copolyesters, and thermoplastic polyamides. In some variations, the flexible layer may include pockets configured as an array (e.g., a grid) corresponding to the arrangement of the light sources. In some variations, the pockets may be configured to cover and/or contain the light sources. For example, referring to FIG. 1, a light therapy device (100) may include a wearable garment, such as a belt (102) configured to be worn on the torso of a subject. The belt (102) may include a material having a first end (104) and a second end (106) that may be attached to each other by a hook-and-loop (e.g., Velcro®) fastener (110). A plurality of light sources (109) including laser diodes (108) may be arranged as an array within the belt (102). The array may include three rows of 7 light sources (109). Although the light sources (109) are shown in FIG. 1 as square shaped and include laser diodes (108) arranged as quartets (four laser diodes per light source (109)), it is understood that any number of light sources and laser diodes may be employed, and that the light sources may be arranged in other ways or patterns, or have other geometries. For example, instead of being square, the light sources may be shaped as a circle, oval, rectangle, triangle, or any other suitable shape. The light sources may include between 1 and 10 laser diodes (109), including 1, 2, 3, 4, 5, 6, 7, 8, 9, or 10 laser diodes. The array of light sources may be arranged in a grid pattern. For example, two or more laser diodes may be arranged along a first axis (e.g., as rows along a horizontal axis) and two or more laser diodes may be arranged along a second axis (e.g., as columns along a vertical axis). The spacing (i.e., distance) between the light sources may also vary. In some variations, the spacing between each light source of the plurality of light sources may be the same. In other variations, the spacing between each light source of the plurality of light sources may be different. In further variations, the spacing between a first portion of the plurality of light sources may be the same, and the spacing between a second portion of the plurality of light sources may be different.

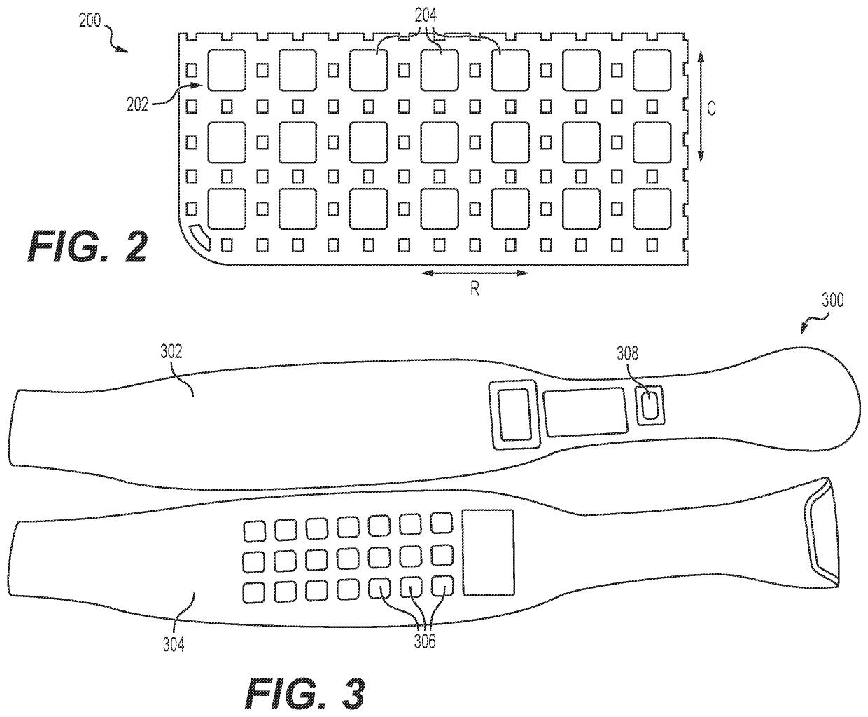

In some variations, a flexible silicone layer (200) as shown in FIG. 2 may be provided over the light sources. Additionally, the silicone layer (200) may be configured as a grid (202), and may include a plurality of pockets (204) configured to contain the light sources. The pockets (204) may be arranged as a plurality of rows running horizontally across the grid (202) in the direction of arrow (R), and a plurality of columns running vertically up and down the grid (202) in the direction of arrow (C). In the example shown in FIG. 2, 21 total pockets are provided by the 3 rows and 7 columns of pockets. The silicone layer may be a transparent layer that allows light from the laser diodes to be transmitted to a target area or tissue (e.g., skin, subdermal tissue, blood vessel, muscle, tendon, ligament, joint) during therapy.

Referring to FIG. 3, another belt (300) including a plurality of material layers is illustrated. In FIG. 3, belt (300) comprises a first backing layer (302) and a second layer (304) including a plurality of openings (306). When a silicone layer is included in the belt (300), similar to silicone layer (200) in FIG. 2, the silicone layer may be disposed over the plurality of light sources (not shown) such that the light sources are contained within pockets of the silicone layer. The silicone covered light sources may then be placed between the first backing layer (302) and the second layer (304) and within openings (306) such that the light sources at least partially extend through the openings (306).

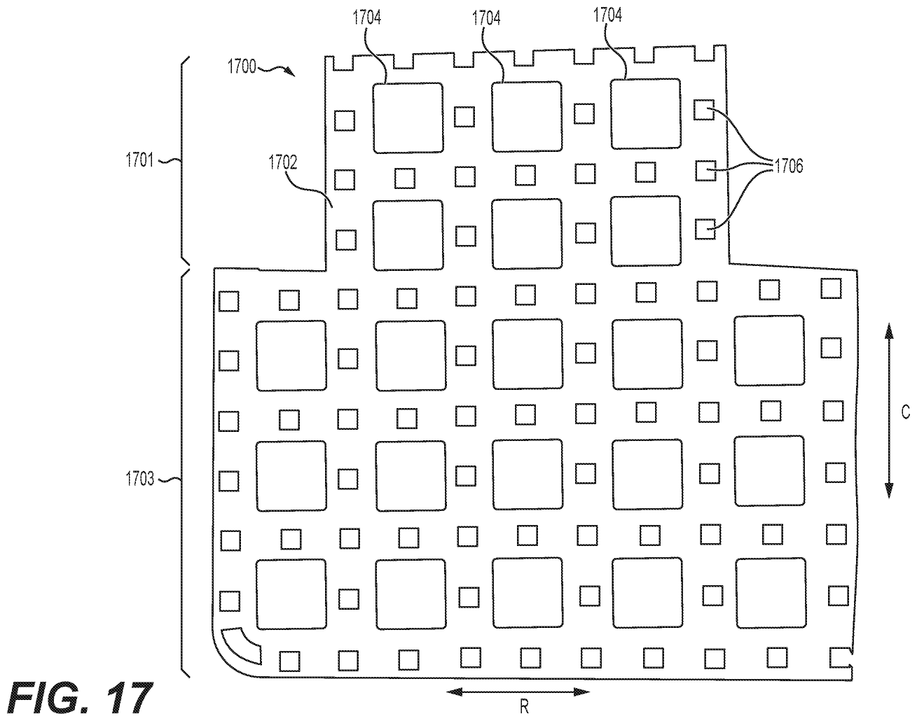

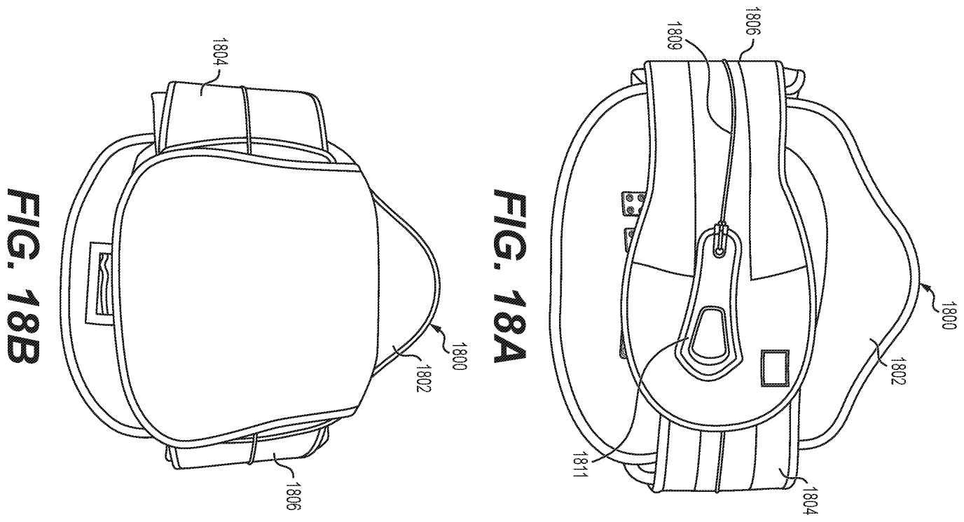

In further variations, a flexible silicone layer (1700) as shown in FIG. 17 may be provided over the light sources. The flexible silicone layer (1700) may be configured as an array (1702), and may include a plurality of pockets (1704) configured to contain the light sources and a plurality of openings (1706). The pockets (1704) and plurality of openings (1706) may be arranged as a plurality of rows running horizontally across the array (1702) in the direction of arrow (R), and a plurality of columns running vertically up and down the array (1702) in the direction of arrow (C). In the example shown in FIG. 17, 21 total pockets (1704) are provided by 2 rows and 3 columns of pockets in a top portion (1701) of the flexible silicone layer (1700), and an additional 3 rows and 5 columns of pockets in a bottom portion (1703) of the flexible silicone layer (1700). As also shown, the openings (1706) may be disposed between the pockets (1704). In some variations, the openings (1706) may be configured to receive a mechanical fastener (e.g., tie, cable, screw, rivet, bolt, button, snap, dowel, pin) such that the flexible silicone layer (1700) may be coupled to the printed circuit board or other layer including electronics and/or light sources, and/or one or more material layers as described herein. Additionally or alternatively, the openings (1706) may be configured to facilitate air flow through the flexible silicone layer (1700), which may increase the comfort of the subject during use by allowing hot air to flow from the subject's body to an external environment.

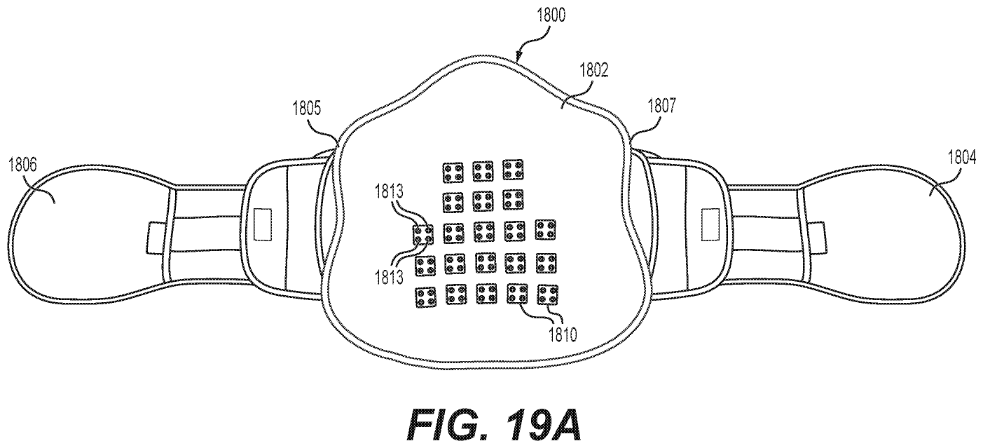

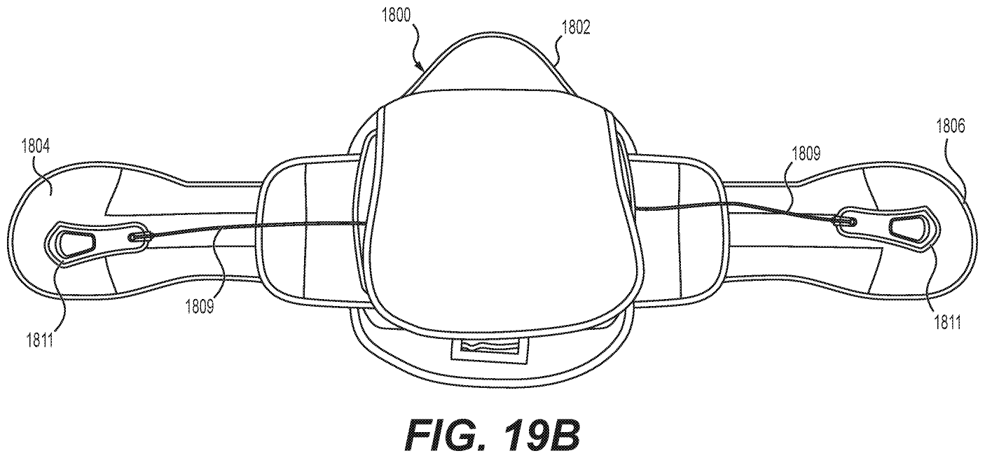

The flexible silicone layer (1700) may be a transparent layer that allows light from the laser diodes to be transmitted during therapy, as previously mentioned. The flexible silicone layer (1700) may be included in a back brace, such as the back brace shown in FIGS. 18A, 18B, 19A, and 19B. As shown, the back brace (1800) may comprise a panel (1802) comprising a plurality of openings, where each opening may be configured to receive a light source (1810). The light sources (1810) may include a quartet of laser diodes as described above, and may be covered by a flexible silicone layer, such as flexible silicone layer (1700). In some variations, the flexible layer, e.g., the flexible silicone layer, may be configured as a sleeve sized and shaped to fit over a backing of the brace, and to hold the plurality of light sources against the backing. The backing of the brace may be made from a material that may be rigid (e.g., plastic materials), flexible (e.g., canvas, cotton, elastic, neoprene), or semi-rigid (e.g., a combination of rigid and flexible materials). The use of a rigid or semi-rigid backing may be beneficial when support of the back (or other body area) and/or limitation of movement (e.g., after injury, fracture, or surgery) of the back (or other body area) is needed in addition to light therapy. The panel may be configured to be removably attached to the back braces in various ways, for example by a hook and loop fastener (e.g., a Velcro® fastener), buckles, snaps, buttons, laces, or a combination thereof.

In other variations, the material may be an elastic material capable of stretching about an additional 5% to about 75%, including all values and sub-ranges therein, beyond the original dimensions of the material while being able to return to the original shape/dimensions of the material when not stretched. For example, the elastic material may be capable of stretching about 5%, about 10%, about 15%, about 20%, about 25%, about 30%, about 35%, about 40%, about 45%, about 50%, about 55%, about 60%, about 65%, about 70%, or about 75% beyond the original dimensions of the material while being able to return to the original shape/dimensions of the material when not stretched. Stretching of the material against the body area surface may better approximate the light sources against that surface and hold them in place.

The wearable garments may be secured on or around body areas by various types of fasteners. The fasteners may allow adjustment of any straps or ends of the wearable garment for fitting around or on the subject. For example, when the material comprises an elongate body having a first end and a second end, a fastener may be configured to removably attach the first end to the second end. Exemplary fasteners may include without limitation, a hook and loop fastener (e.g., a Velcro® fastener), a buckle, a snap, a button, laces, or a combination thereof. In one variation, tension on the material against body areas may be held by fastening the first and second ends of the material while it is stretched. In another variation, the material is not placed under tension or stretched upon fastening first and second ends together. In yet further variations, a flexible or bendable metal band may be included in the wearable garments to help conform and/or secure the garment to the body area.

The size, shape, and type of garment, and the materials used may vary depending on the treatment indication and area of the body for which the device is to be worn. As previously mentioned, the wearable garment may be a belt, band, brace, sleeve, sling, or other type of wrap configured to place the plurality of light sources against the surface, e.g., the skin, of the body area in which the subject is experiencing pain. In some variations, the wearable garment may be a glove, cap, or other item of clothing, e.g., an undergarment, shirt, or pants.

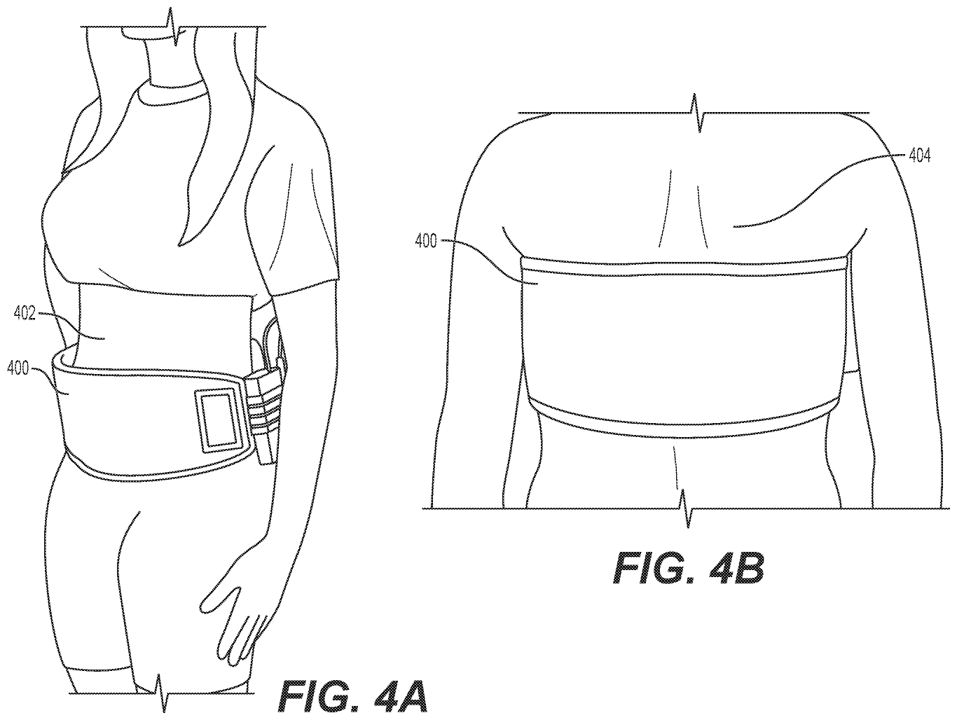

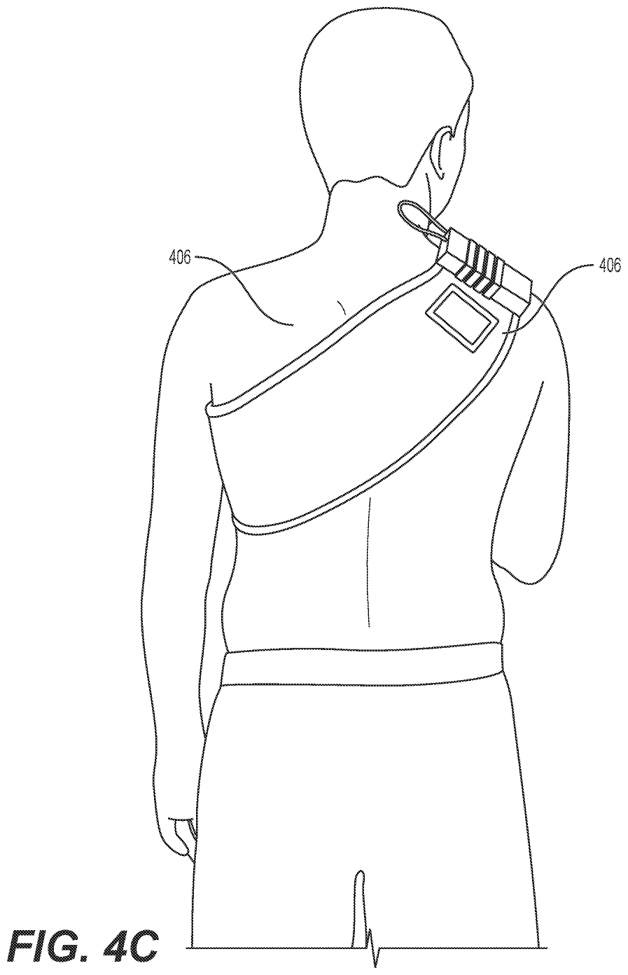

In one variation, the wearable garment may be worn around the torso of the subject. For example, the garment may be worn around or on an upper back, a lower back, an abdomen, a shoulder, or a chest of the subject. Referring to FIGS. 4A to 4C, the wearable garment may include a belt (400) that may be worn around the abdomen (402) (FIG. 4A) or the upper back (404) (FIG. 4B) of a subject. The belt (400) may also be worn in a sling-like fashion across the back (406), as shown in FIG. 4C. In some variations, the belt may be configured as back brace. For example, as shown in FIGS. 18A, 18B, 19A, and 19B, the back brace (1800) may be worn by a subject similar to the belt (400) described in reference to FIGS. 4A to 4C. The back brace (1800) is described in further detail below.

When the wearable garment comprises a belt, the length (horizontal length) of the belt may range from about 127 cm (50 inches) to about 165 cm (65 inches), including all values and sub-ranges therein. For example, the belt length may be about 127 cm, about 130 cm, about 135 cm, about 140 cm, about 145 cm, about 150 cm, about 155 cm, about 160 cm, or about 165 cm. The height (vertical height) of the belt at its may also vary and range from about 10 cm to about 30 cm, including all values and sub-ranges therein. For example, the belt height may be about 10 cm, about 15 cm, about 20 cm, about 25 cm, or about 30 cm.

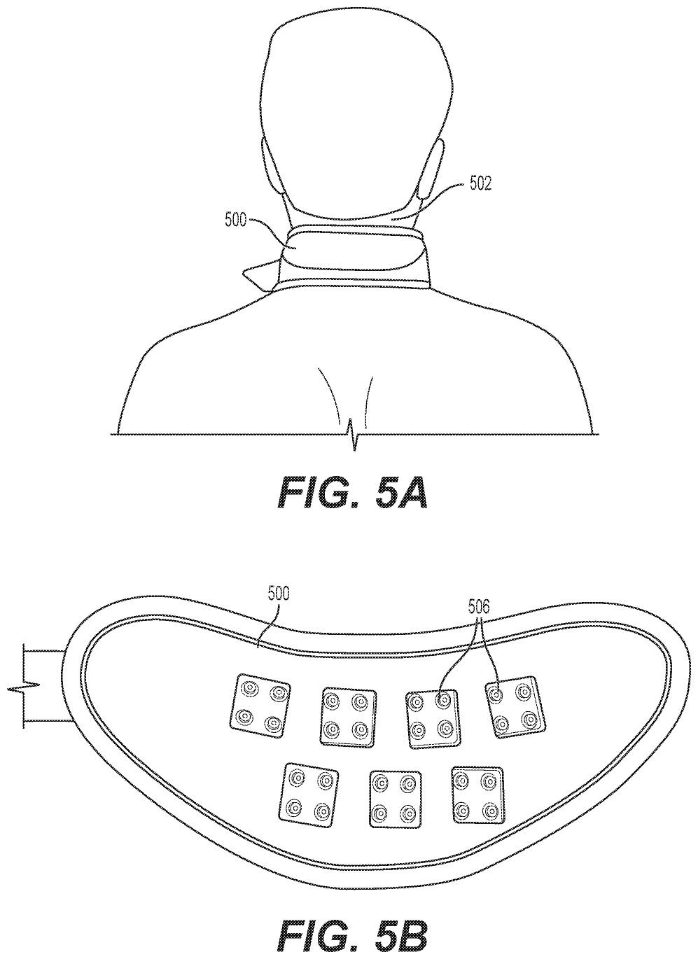

In other variations, the garment (500) may be worn around the neck (502) of the subject, as shown in FIGS. 5A-5C. When the garment is to be worn around the neck, the length and height of the garment may depend on the neck length of the subject. FIGS. 5A and 5B illustrate a neck garment (500) configured to worn by a subject having a normal to short neck. As shown in FIG. 5B, the neck garment (500) may include light sources configured as 7 quartets (506) of laser diodes. In FIG. 5C, a neck garment (504) configured to be worn by a subject having a long neck is shown. The neck garment in FIG. 5C includes light sources configured as 20 quartets (508) of laser diodes. Although square shaped light sources configured as quartets are illustrated in the figures, the plurality of light sources and laser diodes may be configured in other ways, as previously stated. The length of a neck garment may generally range from about 13 cm to about 24 cm, including all values and sub-ranges therein. For example, the length may be about 13 cm, about 13.5 cm, about 14 cm, about 14.5 cm, about 15 cm, about 15.5 cm, about 16 cm, about 16.5 cm, about 17 cm, about 17.5 cm, about 18 cm, about 18.5 cm, about 19 cm, about 19.5 cm, about 20 cm, about 20.5 cm, about 21 cm, about 21.5 cm, about 22 cm, about 22.5 cm, about 23 cm, about 23.5 cm, or about 24 cm. The height of the neck garment may range from about 10.5 cm to about 21.5 cm, including all values and sub-ranges therein. For example, the height may be about 10.5 cm, about 11 cm, about 11.5 cm, about 12 cm, about 12 cm, about 12.5 cm, about 13 cm, about 13.5 cm, about 14 cm, about 14.5 cm, about 15 cm, about 15.5 cm, about 16 cm, about 16.5 cm, about 17 cm, about 17.5 cm, about 18 cm, about 18.5 cm, about 19 cm, about 19.5 cm, about 20 cm, about 20.5 cm, about 21 cm, or about 21.5 cm. In some variations, the neck garment may have a length of about 24 cm, and a height of about 10.5 cm. In other variations, e.g., when the subject has a longer neck, the neck garment may have a length of about 13 cm, and a height of about 21.5 cm.

In some variations, as mentioned above, the wearable garment may be a brace (1800) configured as shown in FIGS. 18A, 18B, 19A, and 19B that may be worn on the back of the subject, such that the plurality of light sources of the brace (1800) may treat a portion of the subject's back. The brace (1800) may comprise a first arm (1804), a second arm (1806), and a panel (1802). The first arm (1804) and second arm (1806) may each extend from the panel (1802). For example, as shown in FIG. 19A, the first arm (1804) may extend from a first end (1805) of the panel (1802) and the second arm (1806) may extend from a second end (1807) of the panel (1802), which may be opposite from the first end (1805). The first and second arms (1804 and 1806, respectively) may each comprise one or more fasteners (e.g., Velcro®, ties, laces, snaps, buttons, etc.), such that the first and second arms (1804, 1806) may be attached together in an adjustable manner. In use, the first and second arms (1804, 1806) may be wrapped around the back of the subject and overlapped for an amount on the abdomen of the subject to securely fit the brace (1800) against the subject. Further adjustment of the size of the brace (1800) may be accomplished using one or more cords (1809) coupled to a tab (1811). For example, FIGS. 18A (front view) and 18B (back view) show the back brace (1800) in a closed configuration, where the first and second arms 1804, 1806 are fastened together. FIGS. 19A (inner side of brace facing the skin of the subject) and 19B (outer surface of the brace) show the brace (1800) in an open configuration, where the first and second arms 1804, 1806 are not attached to each other. The open configuration may facilitate positioning the belt (1800) around the subject, such that the belt (1800) may be transitioned to the closed configuration once the panel (1802) is positioned over the target area or tissue of the body to be treated. In some variations, one or more extension portions may be combined with the first and/or second arms (1804, 1806) to increase the length of the brace (1800) when needed.

The panel (1802) may be configured to provide light therapy as described herein. For example, as shown in FIG. 19A, the panel (1802) may include a plurality of light sources (1810) configured as 21 quartets of laser diodes (1813). In other variations, the panel may comprise between 10 to 50 light sources, where each light source may include any suitable number of laser diodes. For example, the panel may include 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 light sources. The light sources may include four laser diodes (e.g., a quartet), or more laser diodes, e.g., 5, 6, 7, 8, 9, or 10 laser diodes. The panel (1802) may further comprise a support member. The support member may be configured to better conform the panel (1802) to the contour of the lower back (e.g., to improve approximation of the light sources against the surface of the back) and/or house a battery pack (not shown). An exemplary support member (2200) is shown in FIG. 22. The support member (2200) may comprise a support plate (2202) that may define one or more openings (2206). The openings (2206) may be configured to reduce mechanical stresses associated with using and/or handling the garment described herein, as the openings (2206) may allow the support plate (2202) to bend and/or twist without breaking. In some variations, the openings (2206) may be configured to facilitate ventilation therethrough, which may increase subject comfort while wearing the garment. For example, the openings (2206) may facilitate warm air to flow from the subject to an external environment, such that the subject may maintain a stable body temperature. The support member (2200) may further comprise a power cord (2204), which may be coupled to a power source (e.g., battery, external power source).

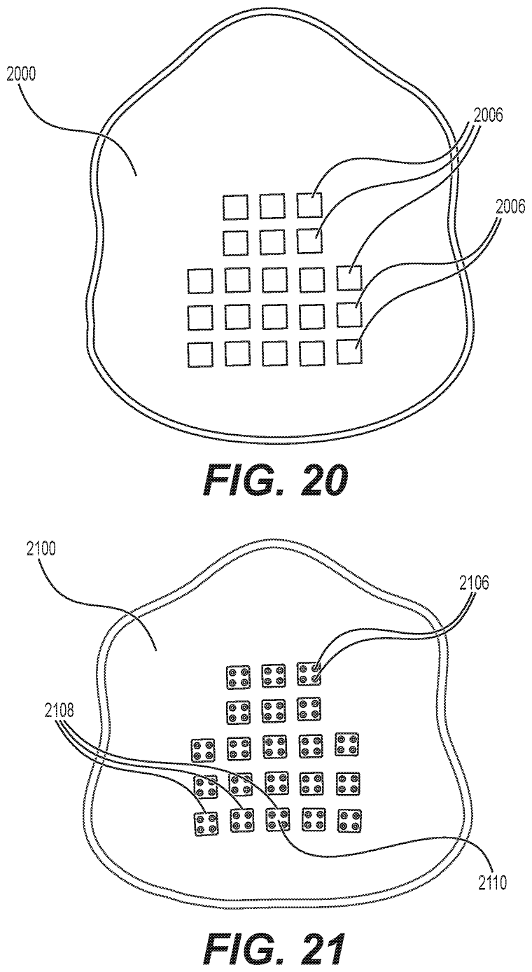

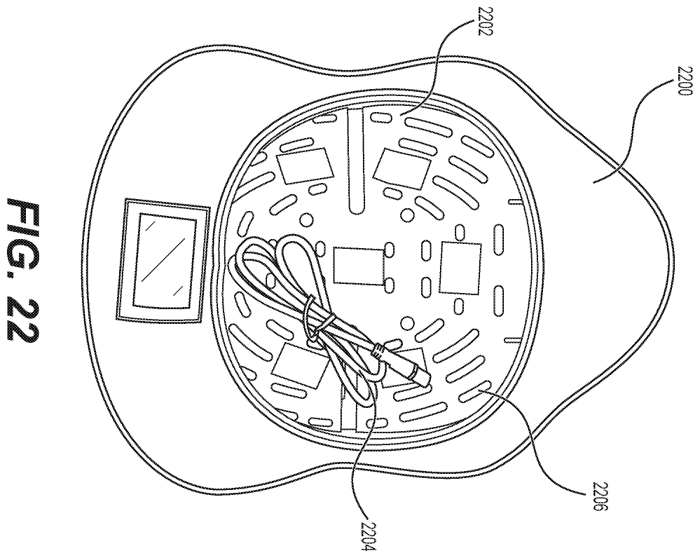

FIGS. 20 and 21 show a panel (2000) alone, detached from a belt. The panel (2000) may define a plurality of openings (2006) that may correspond to a plurality of light sources when placed therein and/or pockets of a silicone layer. For example, as shown in FIG. 21, panel (2000) may include a plurality of light sources (2108) comprising quartets of laser diodes (2106) disposed within the openings (2006). The light sources (2108) may be covered by a silicone layer (2110) that may be at least partially transparent so that laser diodes (2106) may transmit light therethrough.

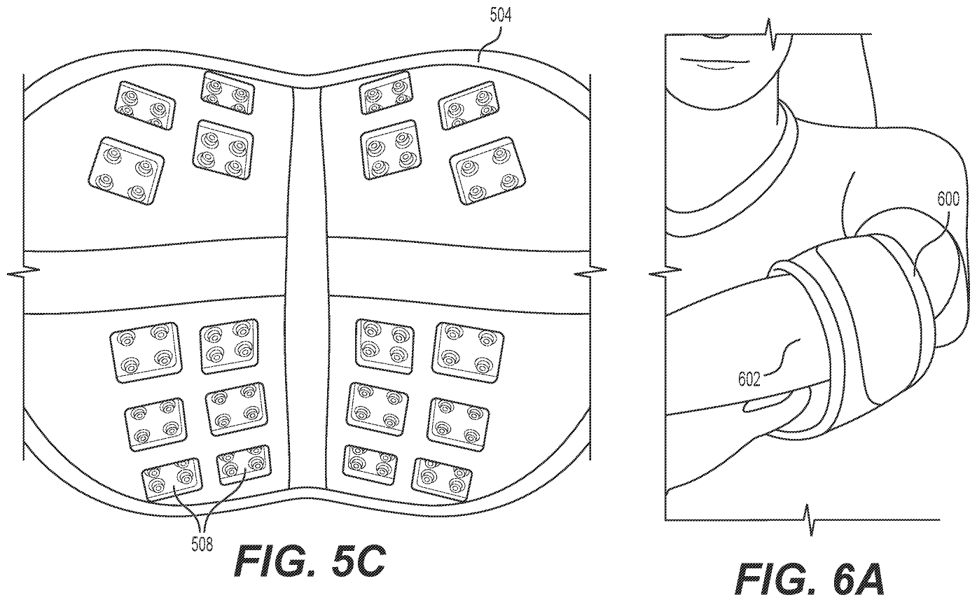

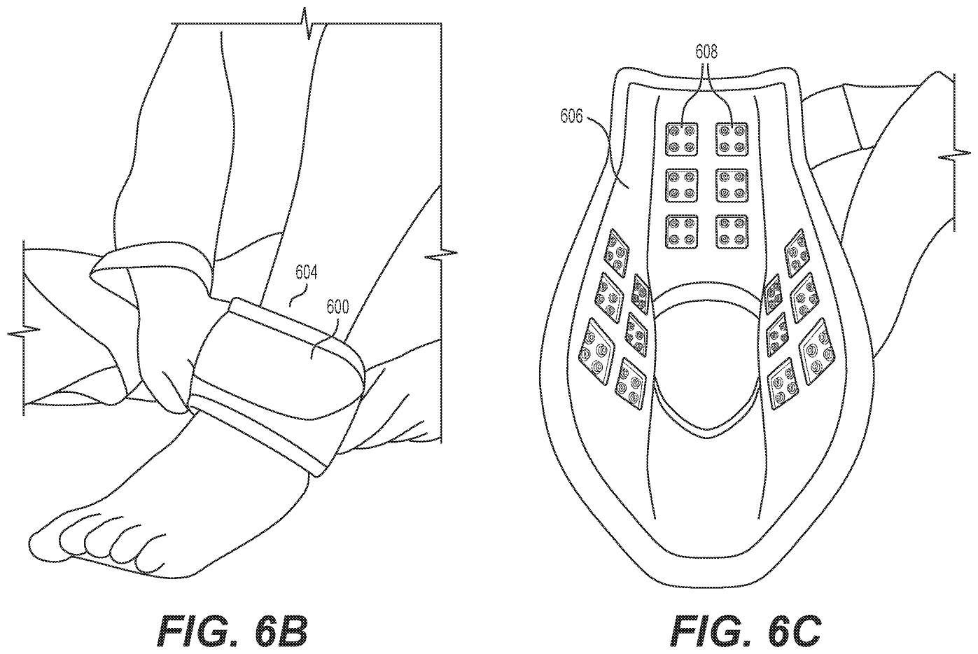

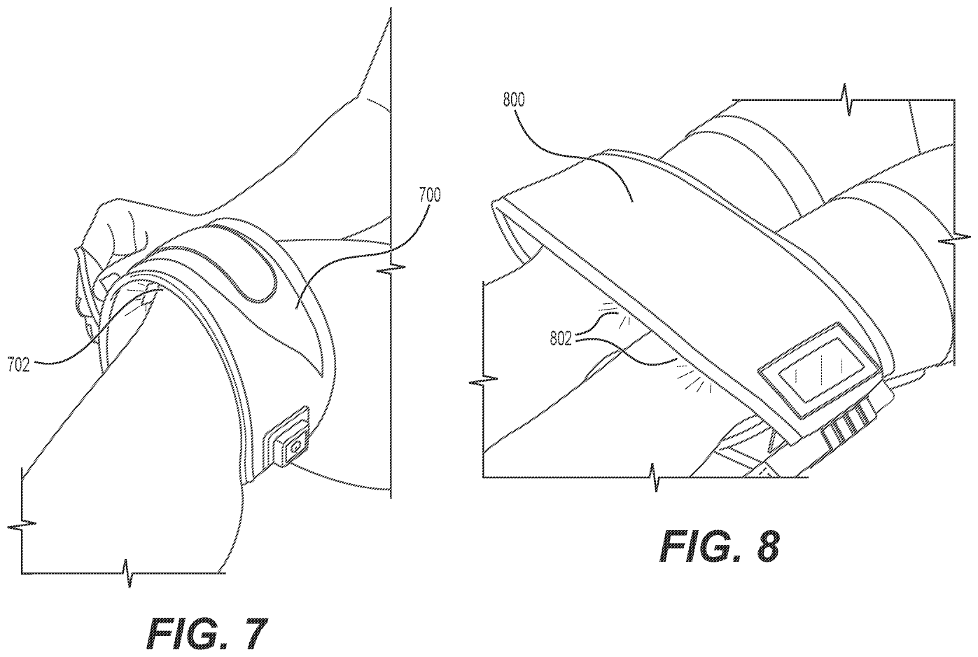

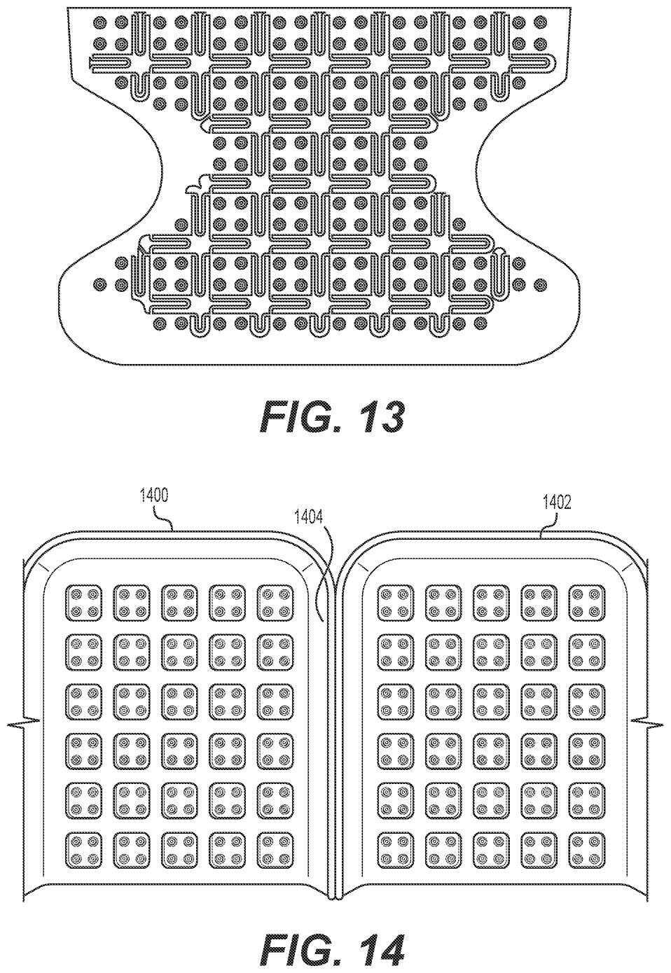

Additionally, the wearable garment may be worn on or around a hand or a joint area, e.g., an elbow, wrist, knee, or ankle of the subject. The body area may also be a limb, e.g., an arm or a leg of the subject, or a joint area, e.g., an elbow, wrist, knee, or ankle of the subject. For example, as shown in FIG. 6A, the wearable garment (600) may be worn around the elbow (602) of a subject, or as shown in FIG. 6B, around the ankle (604) of a subject. In FIG. 6C, another exemplary ankle garment (606) having a plurality of laser diodes arranged as quartets (608) on an inner surface of the garment is provided. The wearable garments (700, 800) may also be worn around a single knee (702) of a subject (FIG. 7), or around both knees (802) of a subject (FIG. 8). In addition to the configurations shown in FIGS. 7 and 8, the knee garment may be shaped as shown in FIG. 13. When used on the hand of a subject, the wearable garment may be configured as shown in FIG. 14 and include a housing having a first portion (1400) attached to a second portion (1402) via, e.g., a hinge (1404) (or other movable joint) configured to fold the portions toward each other such that the portions enclose the hand therein. In other variations, the wearable garment for use with the hands may be configured as a glove including a plurality of light sources.

### Light Sources

The light therapy devices describe herein may include a plurality of light sources configured for PBMT. Any suitable light source may be included in the light therapy devices. For example, light emitting diodes (LEDs) or laser diodes may be used. In some instances, it may be beneficial to employ a plurality of laser diodes. Laser light may be beneficial given that: 1) it is nearly monochromatic, consisting of a narrow range of wavelengths; 2) it is highly directional, traveling in a single direction within a narrow cone of divergence; and/or 3) it is highly coherent and thus capable of penetrating tissue to a deeper level.

Each laser diode of the plurality of laser diodes may be configured to emit light at a wavelength ranging from about 300 nm to about 1200 nm, including all values and sub-ranges therein. For example, the wavelength may be about 300 nm, about 350 nm, about 400 nm, about 450 nm, about 500 nm, about 550 nm, about 600 nm, about 610 nm, about 620 nm, about 630 nm, about 640 nm, about 650 nm, about 660 nm, about 670 nm, about 680 nm, about 690 nm, about 700 nm, about 710 nm, about 720 nm, about 730 nm, about 740 nm, about 750 nm, about 760 nm, about 770 nm, about 780 nm, about 790 nm, about 800 nm, about 810 nm, about 820 nm, about 830 nm, about 840 nm, about 850 nm, about 900 nm, about 950 nm, about 1000 nm, about 1050 nm, about 1100 nm, about 1150 nm, or about 1200 nm. In one variation, it may be useful for the laser diode to emit light at a wavelength between about 600 nm to about 810 nm. In another variation, it may be useful for the laser diode to emit light at a wavelength of 650 nm. In a further variation, it may be useful for the laser diode to emit light at a wavelength of 808 nm.

The wavelength may correspond to a depth of penetration, such that use of the devices described herein may be modified to target specific tissue depths. For example, the penetration depth may be about 0.1 mm to about 5 mm, including all values and sub-ranges therein. For example, the penetration depth may be about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.5 mm, about 2.0 mm, about 2.5 mm, about 3.0 mm, about 3.5 mm, about 4.0 mm, about 4.5 mm, or about 5.0 mm. Generally, the penetration depth may be proportional to the wavelength of the emitted light. For example, a wavelength between about 280 nm to about 320 nm may correspond to a penetration depth of about 0.5 mm, a wavelength between about 320 nm to about 400 nm may correspond to a penetration depth of about 1.0 mm, a wavelength between about 400 nm to about 470 nm may correspond to a penetration depth of about 1.5 mm, a wavelength between about 470 nm to about 550 nm may correspond to a penetration depth of about 2.5 mm, a wavelength between about 550 nm to about 600 nm may correspond to a penetration depth of about 4.0 mm, and a wavelength between about 600 nm to about 1000 nm may correspond to a penetration depth of about 4.0 mm to about 5.0 mm.

In addition to the wavelength, the devices described herein may be modified to adjust the amount of energy (e.g., output voltage) delivered to tissue. For example, each laser diode of the plurality of laser diodes may deliver an amount of energy less than or equal to about 5 mW, e.g., about 5 mW, about 4 mW, about 3 mW, about 2 mW, or about 1 mW.

Additionally, any suitable number of light sources may be included in the light therapy devices. The number of light sources included may depend on such factors as the size and shape of the garment, the particular condition being treated, and/or the body area being treated. The light sources may include one or more laser diodes, as previously described. In some variations, the number of laser diodes included may range from 4 to 220, including all values and sub-ranges therein. For example, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158,160, 162, 164, 166, 168, 170, 172, 174, 176, 178, 180, 182, 184, 186, 188, 190, 192, 194, 196, 198, 200, 202, 204, 206, 208, 210, 212, 214, 216, 218, or 220 laser diodes may be used. When the laser diodes are arranged as quartets, the number of quartets may range from 4 to 50. For example, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 quartets may be included in the light therapy devices. Although described as being arranged in quartets, it is understood that the laser diodes may be grouped in various other numbers. Additionally, the plurality of light sources may have any suitable arrangement. For example, the light sources may be arranged in various patterns. The light therapy device may include light sources arranged in a single row, in a single column, in a circular pattern, in a spiral or helical pattern, in a symmetrical or asymmetrical pattern, or in a checkerboard pattern. The number of light sources employed and their arrangement may differ depending on the type of pain being treated, the amount of pain (e.g., mild, moderate, severe), whether an indication other than pain is being treated, the body area being treated, the size of the area affected by pain or other condition, or a combination thereof. For example, the number of light sources included in a garment to be worn on the neck may include a smaller number of light sources than a garment to be worn on the back, and be arranged as a single row instead of multiple rows.

The light sources may be all the same, or they may include a combination of different light sources. For example, in some variations, the light sources may be all laser diodes, all light emitting diodes (LEDs), or a combination of laser diodes and LEDs. In some variations, when a combination of light sources are employed, an equal number of laser diodes and LEDs may be used. In other variations, the number of laser diodes and LEDs used may not be the same.

In some variations, the light therapy devices may include one or more photodiode proximity sensors. A photodiode proximity sensor is a sensor comprising such a semiconductor device, which operates to detect a distance to an object by measuring the intensity of the light reflected back onto the photodiode by the object. The one or more photodiode proximity sensors may be configured such that the plurality of light sources will only light if they are within a predetermined distance from a body area to be treated, and such that the plurality of light sources will turn off if they are moved more than the predetermined distance away from the body area to be treated.

### Circuit Design

The plurality of light sources described above may be coupled to a printed circuit board (PCB) configured to mechanically support and electrically connect electronic components of the light therapy device. Any suitable number of PCBs may be included in the devices. In some variations, each grouping (e.g., quartet) of laser diodes may be coupled to its own PCB (e.g., a component laden square, as further described below), or a single PCB may support a plurality of light source quartets. A belt, for example, may include 10 columns and 5 rows of laser diode quartets on a single PCB, but an ankle garment may be made using three separate PCBs that are connected to each other, and each having their own number (which may be the same number or a different number) of quartets.

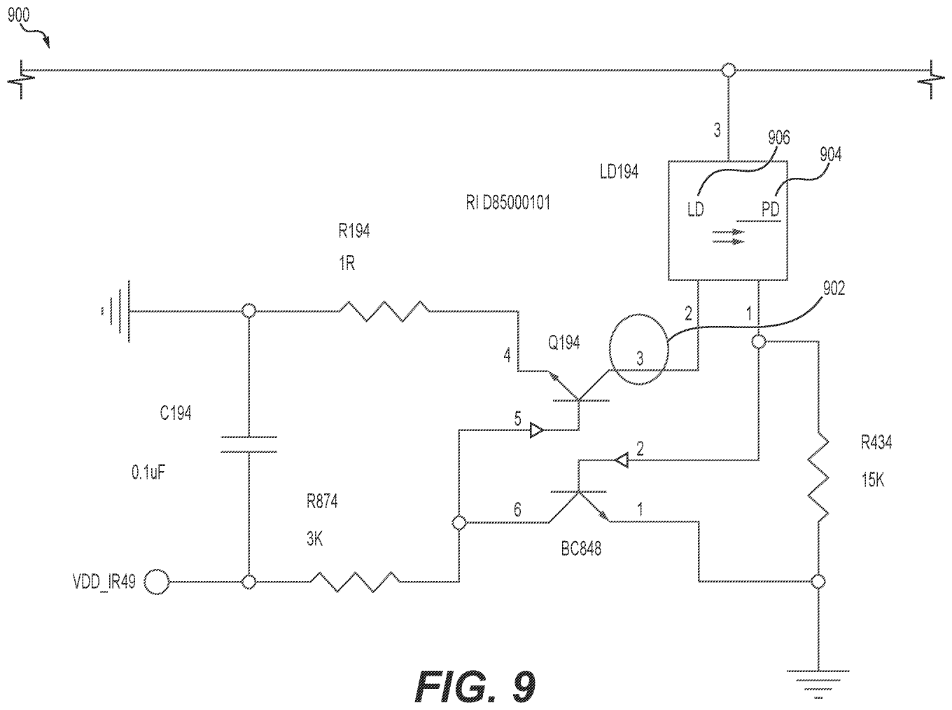

Additionally, the plurality of light sources, e.g., laser diodes, may be powered by an Automatic Power Control (APC) circuit design. The APC circuit may comprise one or more of a resistor, capacitor, and transistor. For example, the APC (900), as shown in FIG. 9, may receive feedback from the third laser diode leg (902) that may be wired to the photodiode (904) contained within the laser diode (906). The photodiode (904) generally measures and monitors the power output of the laser diode (906). The APC circuit may provide a feedback loop whereby the current fed to the laser diode (906) is increased or decreased to maintain the laser output.

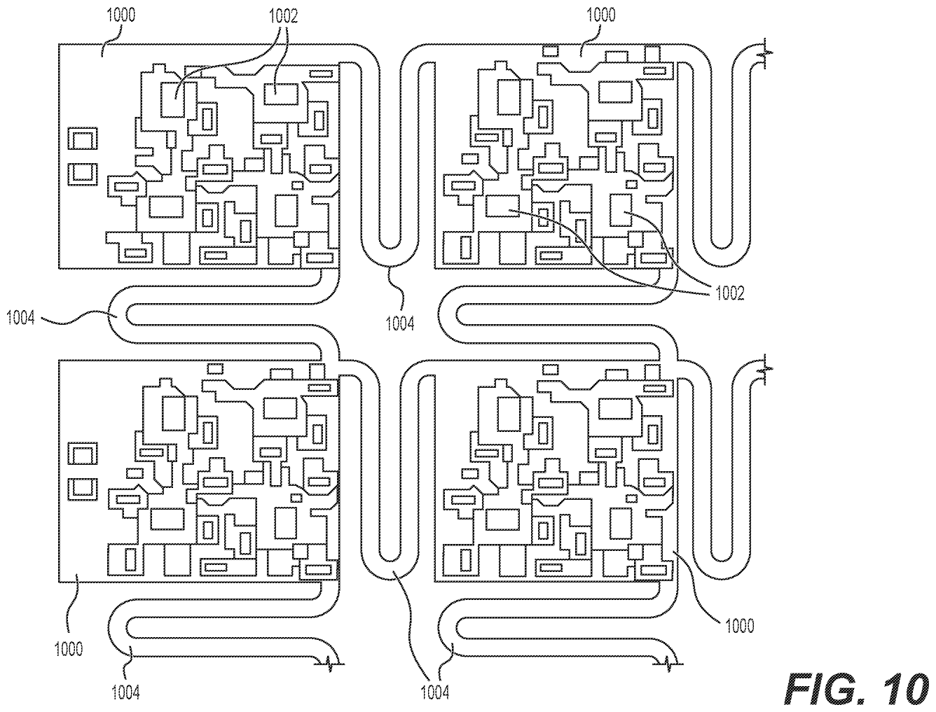

In some variations, as illustrated in FIG. 10, the PCB may be laid out as a series of interconnected component laden squares (1000) containing quartets of individually driven laser diodes (1002) that use the APC circuit design described above. The PCB current flow channels (1004) that connect each diode quartet (1002) may allow for flexibility of the circuit on or within the wearable garment.

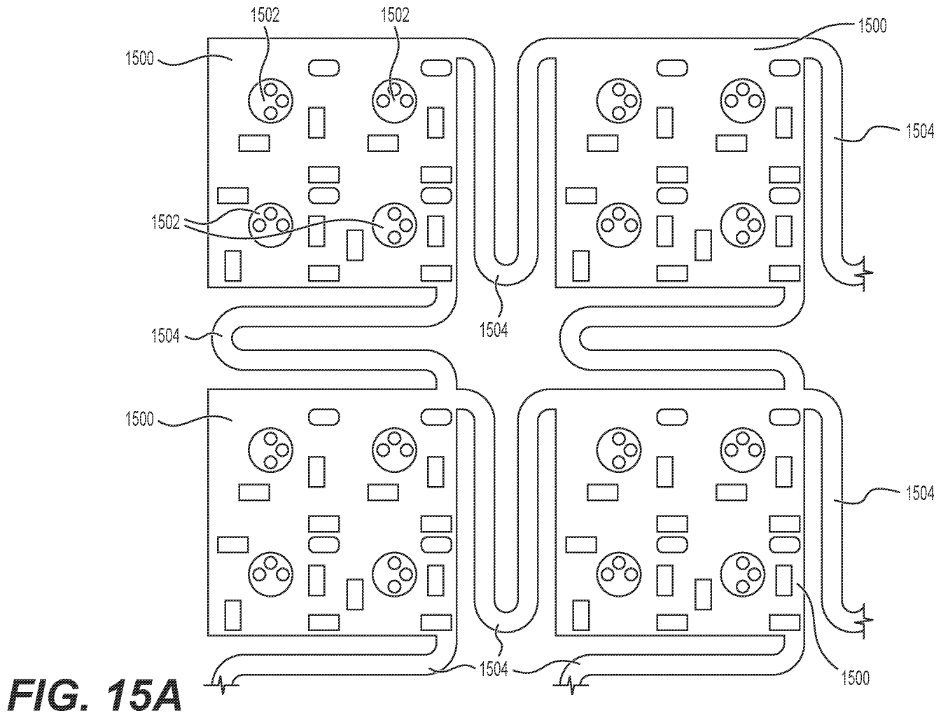

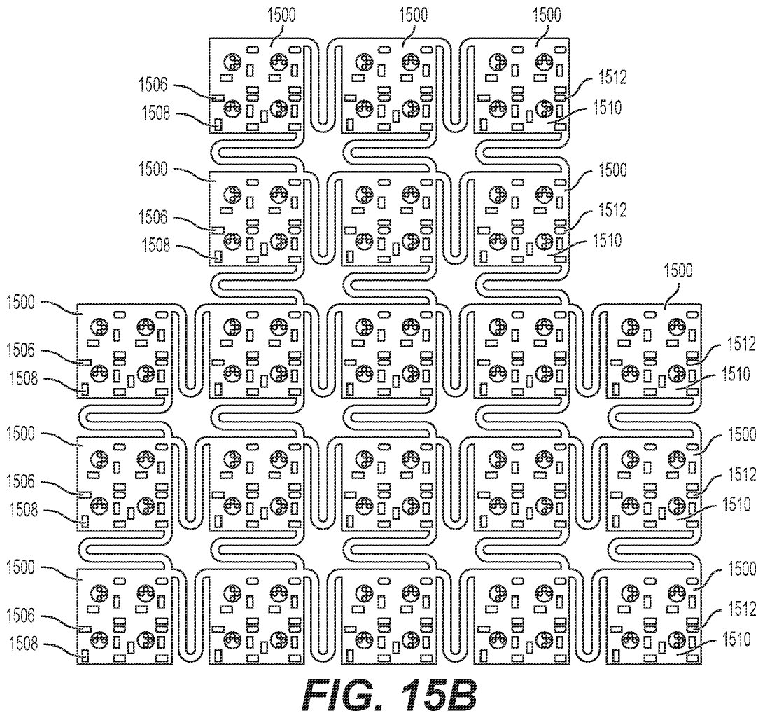

As another example, as shown in FIGS. 15A and 15B, the PCB may comprise a series of component laden squares (1500) that may be interconnected by PCB current flow channels (1504). Each of the component laden squares (1500) may comprise quartets of individually driven laser diodes (1502) that use the APC circuit design described above or an alternative APC circuit design configured to facilitate similar functionality of the laser diodes. Each component laden square (1500) may further comprise a fuse (1506), a resistor (1508), a transistor (1510), and a capacitor (1512). The resistor (1508) may be configured to apply a resistance to an electrical signal flowing through the circuitry described herein, to help avoid damage to the circuitry by the electrical signal. For example, the resistor (1508) may comprise a resistance between about 0.01 kohms (kiloohms) to about 10 kohms (kiloohms), including all values and sub-ranges therein. For example, the resistance may be about 0.01 kohms, about 0.5 kohms, about 1.0 kohms, about 1.5 kohms, about 2.0 kohms, about 2.5 kohms, about 3.0 kohms, about 3.5 kohms, about 4.0 kohms, about 4.5 kohms, about 5.0 kohms, about 5.5 kohms, about 6.0 kohms, about 6.5 kohms, about 7.0 kohms, about 7.5 kohms, about 8.0 kohms, about 8.5 kohms, about 9.0 kohms, about 9.5 kohms, or about 10 kohms. In some variations, the transistor may comprise a switch or gate for the electrical signals, such that current flow may be controlled through the circuitry. For example, the transistor (1510) may comprise a bipolar junction transistor, a field-effect transistor, or a junction field effect transistor. The capacitor (1512) may be configured to store electrical energy. For example, the capacitor (1512) may comprise a capacitance between about 0.01 μF to about 1.0 mF, including all values and sub-ranges therein. For example, the capacitance may be about 0.01 μF, about 0.02 μF, about 0.03 μF, about 0.04 μF, about 0.05 μF, about 1.0 μF, about 10 μF, about 50 μF, about 100 μF, about 150 μF, about 200 μF, about 250 μF, about 300 μF, about 350 μF, about 400 μF, about 450 μF, about 500 μF, about 550 μF, about 600 μF, about 650 μF, about 700 μF, about 750 μF, about 800 μF, about 850 μF, about 900 μF, about 950 μF, or about 1.0 mF (1000 μF.).

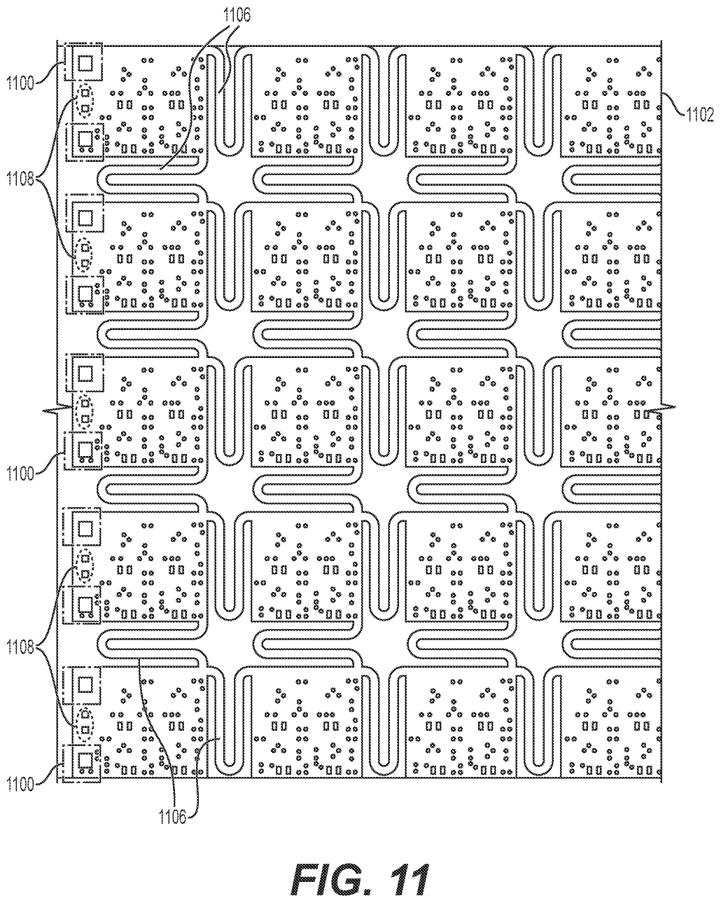

The fuse (1506) may function similarly to the fuse (1108) described in reference to FIG. 11. That is, the fuse (1506) may prevent a broken channel through any row or any other component failure from continuing to pull energy to the electrical components, which may lead to overheating of the light therapy device. The fuse (1506) may be triggered by the power pull and have a fuse tolerance ranging from 1 A to about 3 A, including all values and sub-ranges therein. For example, the fuse tolerance may be about 1 A, about 1.5 A, about 2 A, about 2.5 A, or about 3 A. In some variations, the fuse tolerance may be about 1.5 A. Instead of current, the fuse may be configured to sense temperature and may be triggered when a predetermined temperature has been reached. The fuse may increase the safety of the devices described herein by avoiding risks associated with exposure of the skin to high temperatures.

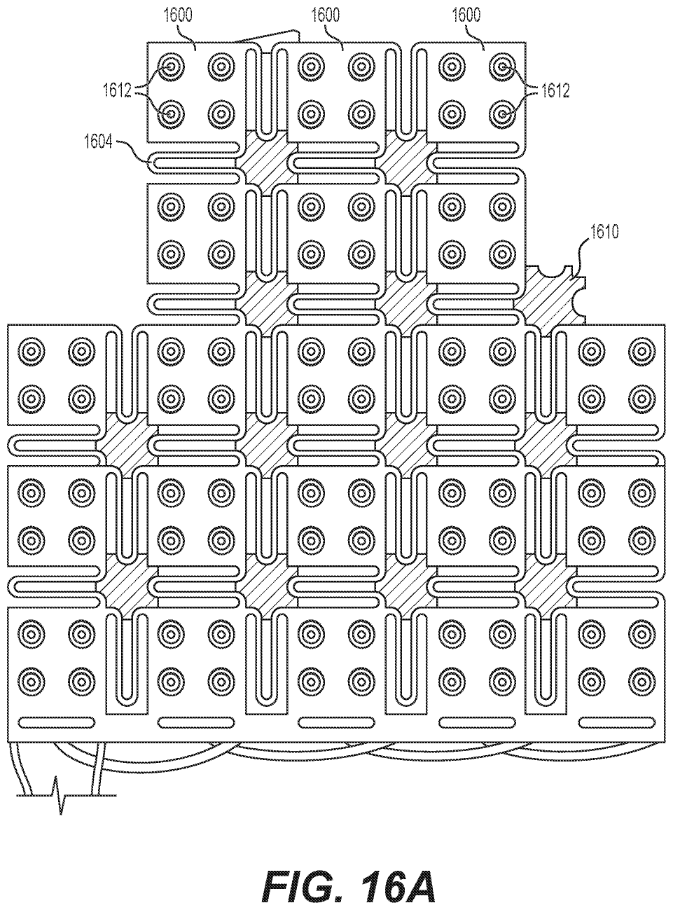

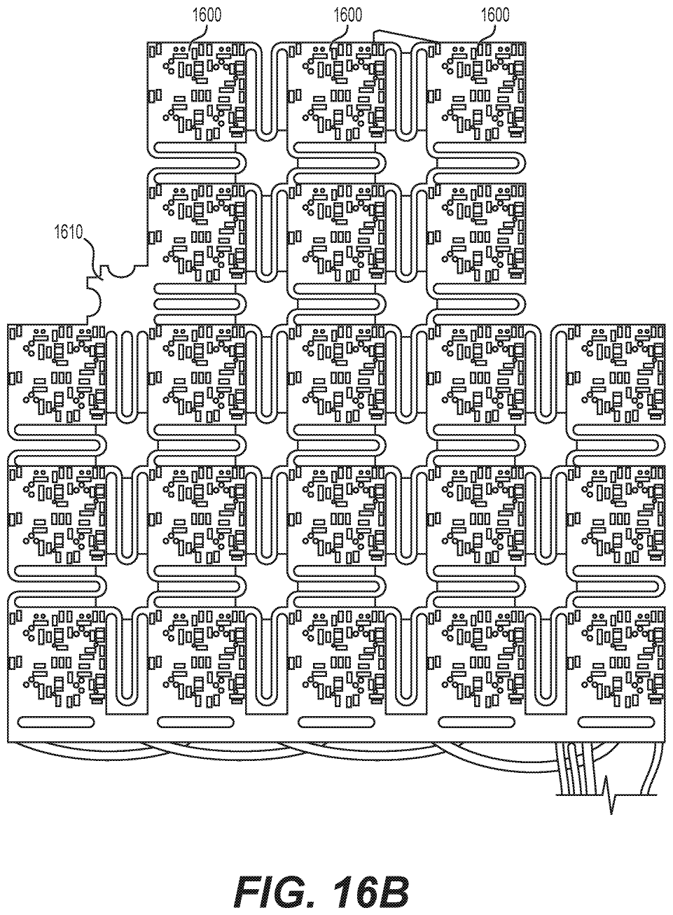

Another exemplary circuit design variation is shown in FIGS. 16A and 16B. As shown, the PCB may comprise a plurality of component laden squares (1600) that may be interconnected. As shown in FIG. 16A, each component laden square (1600) may comprise a quartet of laser diodes (1612). Each laser diode of the quartet of laser diodes (1612) may be arranged proximate to a corner of the component laden square (1600). For example, as shown, the quartet of laser diodes (1612) may form a grid pattern. The component laden squares (1600) may be interconnected by PCB current flow channels (1604), similar to the description provided for element (1504) in reference to FIGS. 15A and 15B. The PCB current flow channels (1604) may have a curved, looped, or serpentine shape. In some variations, the PCB current flow channels (1604) may be supported by pegs (1610). The pegs (1610) may help prevent the PCB current flow channels from bending and/or twisting, which may lead to breakage of the channels.

In general, the laser diodes included in the circuit are low power, e.g., delivering less than or equal to about 5 mW, to avoid the risk of injury due to electricity or heat. An additional safety mechanism to prevent overheating may include multiple power input distribution channels (current flow channels), e.g., one per row at the beginning of each row, that may distribute power flow while stabilizing power consumption. With such a configuration, current feed through any particular point may be reduced, thus minimizing the risk of circuit overheating due to component failure (whether intrinsic or externally induced). For example, as shown in FIG. 11, the safety mechanism may include a plurality of power distribution channels (1106), blue outlined boxes (1100) and a plurality of fuses, red circles (1108) on the PCB (1102). The fuses (1108) may prevent a broken channel through any row or any other component failure from continuing to pull energy to the electrical components, which may lead to overheating of the light therapy device. The fuses (1108) may be triggered by the power pull and have a fuse tolerance of about 1 A, about 2 A, or about 3 A. In some variations, the fuse tolerance may be about 1.5 A. Instead of current, the fuse may be configured to sense temperature and be triggered when a predetermined temperature has been reached.

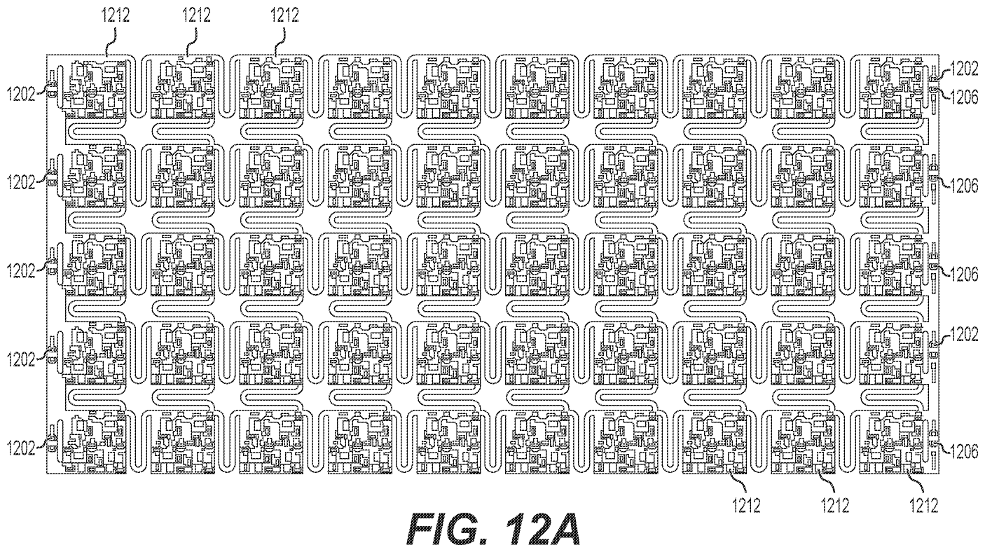

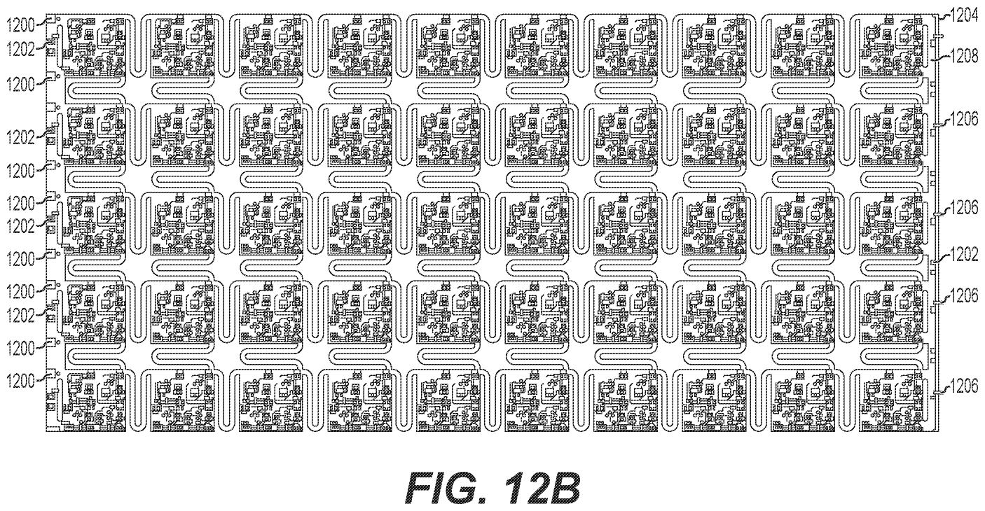

In other variations, as illustrated in FIGS. 12A and 12B, the safety mechanism may include a network of power distribution channels (1200), a network of fuses (1202), and a plurality of bridge jumpers (1206). The bridge jumpers (1206) may be included to prevent a broken channel through any row from continuing to pull energy to the electrical components (i.e., the bridge jumpers may allow continued delivery of current to the components in each row in case of any broken channels that prevent current delivery from the main power input side). The bridge jumpers (1206) may comprise soldering joints or pads. Additionally, the fuses (1202) that are next to the bridge jumpers (1206) may also help prevent component failure from causing overheating of the device resulting from the failure. As an added precaution, the fuses (1202) mounted to each PCB (1212) and associated with the current distribution channels (1200) on each PCB (1212), may be configured to provide overcurrent protection and stop power flow upon encountering a current surcharge. Similar features (e.g., plurality of bridge jumpers) may be included with the variations shown in FIGS. 15A to 15B, or any other variation described herein.

### Power Supply and Controller

The light therapy devices described herein are generally coupled to a portable power supply configured to provide power to the plurality of light sources. The power supply may be a battery pack comprising one or more batteries. In one variation, the battery pack may be rechargeable. For example, the battery pack may be configured to receive an input voltage of between about 100 AC to about 240 AC, including all values and sub-ranges therein. For example, the input voltage may be about 100 AC, about 110 AC, about 120 AC, about 130 AC, about 140 AC, about 150 AC, about 160 AC, about 170 AC, about 180 AC, about 190 AC, about 200 AC, about 210 AC, about 220 AC, or about 240 AC. In some variations, the input voltage may be about 120 AC. The battery pack may be configured to provide an output voltage to the plurality of light sources (e.g., laser diodes). The output voltage may be about 1 VDC to about 10 VDC, including all values and sub-ranges therein. For example, the output voltage may be about 1 VDC, about 2 VDC, about 3 VDC, about 4 VDC, about 5 VDC, about 6 VDC, about 7 VDC, about 8 VDC, about 9 VDC, or about 10 VDC. In some variations, the output voltage may be about 5 VDC.

A display may be included at one end of the battery pack configured to show on/off status of the device, charge status of the battery, and/or the duration of treatment time. A cord may extend from the battery and have an end configured to plug into a connector on the wearable garment that feeds power to the light sources. In some variations, the wearable garment may include a retainer, e.g., a compartment, pocket, or strap for holding the battery back. For example, referring to FIG. 1, wearable garment (102) may include a strap (112) for securing the battery pack (114) to the wearable garment (102). A cord (116) may extend from the battery pack (114) and be configured to couple with a connector (308) (shown in FIG. 3). An alternative variation of a cord (2204) is shown in FIG. 22. The cord (2204) may be configured to couple to a power source, such as a battery pack (not shown). The cord (2204) may be coupled to one or more laser diodes, such that power provided by the power source may be transmitted to the one or more laser diodes. The cord (2204) may be covered by one or more portions of the wearable garments described herein, or in some variations, a portion of the cord (2204) may protrude from the wearable garment. In some variations, the cord (2204) may be accessible by a subject, such as to allow the subject to couple and/or decouple the cord (2204) to a power source. Additionally or alternatively, the wearable garment may not be configured to receive and run on battery power and instead include a cord that may be plugged into an electrical outlet.

A controller may also be included in the battery pack or be provided on or within the wearable garment and coupled to the battery pack. The controller may be configured to control the power supplied to the light sources of the light therapy device. For example, the controller may control the duration of light therapy. In some variations, the controller may control each light source or each quartet of light sources independently, allowing different patterns to be made with the light sources. In this manner, particular light source patterns may be customized for a particular treatment, or to target a particular area of the body.

### Methods

Methods for delivering light therapy to a subject in need thereof are also described herein. The method may generally include positioning a light therapy device on a body area of the subject, where the light therapy device may include a plurality of light sources electrically coupled to a printed circuit board, and delivering light from the plurality of light sources for a duration of time to the body area. As described above, the printed circuit board may have a first end, and the plurality of light sources configured as an array having multiple rows, where each row of the multiple rows comprises a power distribution channel at the first end of the printed circuit board. The light therapy devices may further include a safety mechanism comprising a fuse associated with the power distribution channel, and a network of bridge jumpers on the printed circuit board configured to stabilize current flow to the plurality of light sources.

The light from the plurality of light sources may be used for various indications. For example, the light may be used to treat or manage pain. When used to treat or manage pain, the pain may be mild, moderate, or severe pain. The pain may also be due to various conditions, such as but not limited to, arthritis, inflammation, injury to the body area, or a combination thereof. In some variations, the light is delivered from a plurality of laser diodes.

When treating pain, the duration of time in which light from each of the plurality of light sources may be delivered to a body area may range from about 5 minutes to about 30 minutes, including all values and sub-ranges therein. For example, the duration of time may be about 5 minutes, about 10 minutes, about 15 minutes, about 20 minutes, about 25 minutes, or about 30 minutes. In some instances, it may be beneficial for the duration of time to be about 12 minutes. The duration of time may be pre-programmed into the light therapy device, and in some instances may also be manually adjusted after therapy has started. In other instances, the duration of time may be manually input by the subject.

The light therapy devices may be placed on, and secured to or over, various target body areas or target tissues to treat, e.g., pain or other conditions, affecting those body areas or tissues. For example, the body area may be a neck or a torso of the subject, a back (e.g., an upper back or a lower back), an abdomen, a shoulder, or a chest of the subject. The body area may also be a limb, e.g., an arm or a leg of the subject, or a joint area, e.g., an elbow, wrist, knee, or ankle of the subject. When targeting a specific tissue, the light may be used to treat tissues such as skin, subdermal tissues, blood vessels, muscles, tendons, ligaments, and/or joints.

Delivery or administration of light from the plurality of light sources for pain and other indications may occur once a day or multiple times a day. When delivered or administered multiple times a day, the time interval between treatment sessions may range between about 15 minutes to about 30 minutes, including all values and sub-ranges therein. For example, the time interval between session may be about 15 minutes, about 20 minutes, about 25 minutes, or about 30 minutes. In some variations, the time interval between sessions ranges between about 1 hour and about 12 hours. For example, the time interval may be about 1 hour, about 2 hours, about 3 hours, about 4 hours, about 5 hours, about 6 hours, about 7 hours, about 8 hours, about 9 hours, about 10 hours, about 11 hours, or about 12 hours. The light therapy may also repeated once a day for a plurality of days, or delivered multiple times a day for a plurality of days. For example, light therapy may be delivered for 12 minutes once a day, twice a day, or three times a day for one or more days. The plurality of days may or may not be consecutive. When delivered multiple times a day and/or for a plurality of days, the duration of each light therapy session may be the same or may be different.

In use, the light therapy device may be secured to the body area of a subject to be treated, and power supplied to the light sources by pressing a button or moving a switch on the battery pack or controller. The light sources may deliver therapy for about 5 minutes to about 30 minutes, as previously described. Given that the light therapy device is a portable, wearable device, the subject may be able to walk around during the process and perform other tasks.

Each light source of the plurality of light sources may be configured to emit light at a wavelength ranging from 300 nm to about 1200 nm, including all values and sub-ranges therein. For example, the wavelength may be about 300 nm, about 350 nm, about 400 nm, about 450 nm, about 500 nm, about 550 nm, about 600 nm, about 610 nm, about 620 nm, about 630 nm, about 640 nm, about 650 nm, about 660 nm, about 670 nm, about 680 nm, about 700 nm, about 710 nm, about 720 nm, about 730 nm, about 740 nm, about 750 nm, about 760 nm, about 770 nm, about 780 nm, about 790 nm, about 800 nm, about 810 nm, about 820 nm, about 830 nm, about 840 nm, about 850 nm, about 900 nm, about 950 nm, about 1000 nm, about 1050 nm, about 1100 nm, about 1150 nm, or about 1200 nm. In one variation, it may be useful for the laser diode to emit light at a wavelength between about 600 nm to about 810 nm. In another variation, it may be useful for the laser diode to emit light at a wavelength of 650 nm. In a further variation, it may be useful for the laser diode to emit light at a wavelength of 808 nm. The light source may be a laser diode or a LED.

During treatment, the light sources may emit monochromatic red light. Monochromatic red light may increase blood circulation, improve cellular activity, and reverse the normal deterioration of cells. The improved respiration at the cellular level may increase ATP, modulate reactive oxygen species, and cause release of nitric oxide, which in turn may help improve pain.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated.

1. A light therapy device configured to be worn by a subject comprising:
a plurality of light sources electrically coupled to a printed circuit board, the printed circuit board having a first end, and the plurality of light sources configured as an array having multiple rows, wherein each row of the multiple rows comprises a power distribution channel at the first end of the printed circuit board; and
a safety mechanism comprising a fuse associated with the power distribution channel, and a network of bridge jumpers on the printed circuit board configured to stabilize current flow to the plurality of light sources.

2. The light therapy device of claim 1, wherein the plurality of light sources comprises a plurality of laser diodes.

3. The light therapy device of claim 2, wherein each laser diode of the plurality of laser diodes is configured to emit light at a wavelength ranging from 600 nm to 810 nm.

4. The light therapy device of claim 3, wherein each laser diode of the plurality of laser diodes is configured to emit light at a wavelength of 650 nm.

5. The light therapy device of claim 1, further comprising a power source configured to provide power to the plurality of light sources.

6. The light therapy device of claim 5, wherein the power source comprises a battery pack.

7. The light therapy device of claim 6, wherein the battery pack is rechargeable.

8. The light therapy device of claim 1, further comprising a controller configured to control the duration of light therapy.

9. The light therapy device of claim 1, further comprising a silicone layer disposed over the plurality of light sources.

10. The light therapy device of claim 1, wherein the plurality of light sources are disposed between one or more layers of a flexible material.

11. The light therapy device of claim 10, wherein the flexible material comprises polyurethane, polyester, styrene-butadiene rubber, or a combination thereof.

12. The light therapy device of claim 10, wherein the flexible material comprises an elongate body having a first end and a second end, and a fastener configured to removably attach the first end to the second end.

13. The light therapy device of claim 12, wherein the fastener comprises a hook and loop fastener, a buckle, a snap, a button, laces, or a combination thereof.

14. The light therapy device of claim 10, further comprising a malleable metal band disposed between the one or more layers of the flexible material.

15. The light therapy device of claim 1, wherein the plurality of light sources are disposed within or on a wearable garment.

16. The light therapy device of claim 15, wherein the wearable garment is a belt, band, brace, sling, sleeve, or wrap.

17. The light therapy device of claim 15, wherein the wearable garment is configured to be worn on a back of the subject.

18. The light therapy device of claim 15, wherein the wearable garment is configured to be worn on a torso of the subject.

19. The light therapy device of claim 15, wherein the wearable garment is configured to be worn on an upper back of the subject.

20. The light therapy device of claim 15, wherein the wearable garment is configured to be worn on a lower back of the subject.

21. The light therapy device of claim 15, wherein the wearable garment is configured to be worn on a neck, elbow, wrist, knee, or ankle of the subject.

22. The light therapy device of claim 1, wherein at least a portion of the device is configured to conform to a body surface.

23. The light therapy device of claim 1, wherein at least a portion of the device comprises an elastomeric material.

24. The light therapy device of claim 23, wherein the elastomeric material comprises styrene-butadiene rubber.

25. The device of claim 2, wherein the plurality of laser diodes comprises 16 to 220 laser diodes.

26. The device of claim 25, wherein the plurality of laser diodes comprises 84 laser diodes.

27. A method of treating pain in a subject in need thereof comprising:
positioning a light therapy device on a body area of the subject, the light therapy device comprising:
a plurality of light sources disposed therein and electrically coupled to a printed circuit board, the printed circuit board having a first end, and the plurality of light sources configured as an array having multiple rows, wherein each row of the multiple rows comprises a power distribution channel at the first end of the printed circuit board; and
a safety mechanism comprising a fuse associated with the power distribution channel, and a network of bridge jumpers on the printed circuit board configured to stabilize current flow to the plurality of light sources; and
delivering light from the plurality of light sources for a duration of time to the body area afflicted with pain.

28. The method of claim 27, wherein the duration of time ranges from about 5 minutes to about 30 minutes.

29. The method of claim 28, wherein the duration of time is about 12 minutes.

30. The method of claim 27, wherein the duration of time is pre-programmed into the light therapy device.

31. The method of claim 27, wherein the plurality of light sources comprises a plurality of laser diodes.

32. The method of claim 31, wherein each laser diode of the plurality of laser diodes is configured to emit light at a wavelength ranging from 600 nm to 810 nm.

33. The method of claim 31, wherein each laser diode of the plurality of laser diodes is configured to emit light at a wavelength of 650 nm.

34. The method of claim 27, wherein the pain is due to arthritis, inflammation, injury to the body area, or a combination thereof.

35. The method of claim 27, wherein the body area is a torso of the subject.

36. The method of claim 27, wherein the body area is an upper back, an abdomen, a shoulder, or a chest of the subject.

37. The method of claim 27, wherein the body area is a lower back of the subject.

38. The method of claim 27, wherein the body area is a neck of the subject.

39. The method of claim 27, wherein the body area is a limb of the subject.

40. The method of claim 39, wherein the limb is an arm or a leg of the subject.

41. The method of claim 27, wherein the body area is a joint area of the subject.

42. The method of claim 41, wherein the joint area is an elbow, wrist, knee, or ankle of the subject.

43. The method of claim 27, wherein delivering light from the plurality of light sources occurs once a day.

44. The method of claim 27, wherein delivering light from the plurality of light sources occurs multiple times a day.

45. The method of claim 44, wherein a time interval between the multiple deliveries of light ranges from about 15 minutes to about 30 minutes.

46. A method of treating pain in a subject in need thereof comprising:
positioning a light therapy device on a body area of the subject, the light therapy device comprising a plurality of laser diodes, wherein each laser diode of the plurality of laser diodes is configured to emit light at a wavelength of 650 nm; and
delivering light from the plurality of laser diodes for 12 minutes to the body area afflicted with pain.

47. The method of claim 46, wherein the light therapy device is configured as a belt with a hook and loop fastener.

48. The method of claim 46, wherein the body area is a lower back of the subject.

49. The method of claim 46, wherein the pain is due to arthritis, inflammation, injury to the body area, or a combination thereof.
